(19) United States
(12) Patent Application Publication
(10) Pub. No.: US 2005/0165313 A1
(43) Pub. Date: Jul. 28, 2005

(54) Transducer assembly for ultrasound probes

(76) Inventors: Jacquelyn M.B. Byron, Arlington, MA (US); Heather Beck Knowles, Devens, MA (US)

Correspondence Address: PHILIPS INTELLECTUAL PROPERTY & STANDARDS, P.O. BOX 3001, BRIARCLIFF MANOR, NY, 10510, US

(21) Appl. No.: 11/040,057

(22) Filed: Jan. 21, 2005

Related U.S. Application Data
(60) Provisional application No. 60/539,300, filed on Jan. 26, 2004.

(51) Int. Cl. A61B 8/14

(52) U.S. Cl. 600/459

(57) ABSTRACT

Transducer assembly for an ultrasound probe including a transducer array having transducer elements and an acoustic window attached directly to the transducer array such that the transducer array and the acoustic window form an integral unit. The acoustic window includes a layer of elastomer optionally covered on upper and lower surfaces by impervious polymer layers. An ultrasound probe, such as a transesophageal echocardiographic probe, a transnasal probe, a transthoracic probe, an intracavity probe and an intraoperative probe, including the transducer assembly in a cavity of the housing or nose is also disclosed.

### CROSS REFERENCE TO RELATED CASES

Applicants claim the benefit of Provisional Application Ser. No. 60/539,300, filed 26 Jan. 2004.

### FIELD OF THE INVENTION

The present invention relates generally to ultrasound probes and more particularly to a transducer assembly for ultrasound probes including an integral acoustic window.

### BACKGROUND OF THE INVENTION

Ultrasound probes are commonly used for imaging internal body parts. One type of ultrasound probe includes a transducer array mounted in a body and having an active surface oriented toward an acoustic window connected to the body and which is separate from the transducer array. The body is usually made of hard plastic and is referred to as a housing for transesophageal echocardiographic or TEE probes and as a nose for transthoracic or intracavity probes. Thus, while the term housing will be used hereinafter in connection with the description of the invention below, it should be understood that when applied to ultrasound probes other than TEE probes, it denotes the nose or equivalent body thereof.

TEE probes are used for viewing planar ultrasound images of a patient's heart from inside of the patient's esophagus. The tip of a typical prior art TEE probe houses a rotatable transducer array. Rotation of the transducer array causes a corresponding rotation of the image plane about an image axis. Once the TEE probe is inserted in the esophagus, rotation of the transducer array is controlled at a remote distance from a tip of the probe.

A drawback of such prior art TEE probes is that it is necessary to rotate the transducer array to obtain multi-planar images of the object being examined in the patient's body. Accordingly, the probe must include associated structure to provide for rotation of the transducer array during an examination. This associated structure imposes size and space constraints on the probe.

A portion of a prior art TEE probe is shown in FIG. 8 and includes a support 100, an acoustic window 102 fixed to the support 100 and a transducer array 104 rotatable on a gear 106 relative to the support 100 and the acoustic window 102. Transducer array 104 is sometimes referred to in the art as an acoustic stack assembly. The transducer array 104 includes a layer of piezoelectric material 108, one or more acoustic matching layers 110 adjacent the active surface of the piezoelectric material 108 and a backing layer 112 on the reverse side of the piezoelectric material 108. A heatsink 114 supports the backing layer 112 on the gear 106. A lens 116 is formed over the acoustic matching layer(s) 110. Between an inner surface of the acoustic window 102 and an outer surface of the lens 116, an oil/lubrication layer 118 is provided. The acoustic window 102 is exposed to the environment through an opening formed in the housing 120 of the probe.

During use of a TEE probe, an outer surface of the acoustic window 102 is exposed to the surrounding environment and thus the acoustic window 102 serves as the interface between the transducer array 104 and the surrounding environment. Ultrasonic waves generated by the transducer array 104 pass through the acoustic window 102 in their path toward and from the body parts being imaged. The acoustic window 102 also contacts the patient to ensure optimal acoustic conditions.

Various factors are considered when selecting materials from which to construct the acoustic window. It is desired that the acoustic window is formed from a material that has an acoustic impedance which matches, or at least closely approximates, that of the human body part being imaged, such as tissue of the human body. Acoustic impedance is based on the elasticity, mass density and speed of sound of the material. Additional characteristics of the material include acceptable mechanical and electrical performance, biocompatibility, chemical resistance, low attenuation and stability to ultraviolet rays.

### OBJECTS AND SUMMARY OF THE INVENTION

It is an object of the present invention to provide a new and improved transducer assembly including an integral acoustic window and an ultrasound probe including the same.

It is another object of the present invention to provide a new and improved ultrasound probe including a transducer assembly which does not have to rotate to obtain multi-planar, volumetric three-dimensional images.

In order to achieve these objects and others, a transducer assembly for an ultrasound probe in accordance with the invention includes a transducer array comprising a plurality of transducer elements and an acoustic window attached directly to the transducer array such that the transducer array and the acoustic window form an integral unit. The transducer elements may be electronically controlled to provide the multi-planar, volumetric three-dimensional images.

The transducer array may include a layer of piezoelectric material having an active surface and defining the transducer elements, an acoustic matching section having a lower surface adjacent the active surface of the piezoelectric material and an opposed upper surface and a backing layer arranged on an opposite side of the piezoelectric material from the acoustic matching section. The acoustic window would then be attached to the upper surface of the acoustic matching section.

One particular form of an acoustic window includes a layer of elastomer having opposed surfaces, a first layer of an impervious polymer arranged on one surface of the elastomer layer and a second layer of an impervious polymer arranged on the other surface of the elastomer layer. As such, the elastomer layer is sandwiched between the first and second impervious polymer layers. With this construction, a lower surface of one polymer layer is attached to the upper surface of the transducer array and an upper surface of the other polymer layer defines an exposed surface of the transducer assembly.

The elastomer may be PEBAX™ while the polymer layers may be thin polymer films made from a material having a negligible acoustic impact such as polyethylene, Mylar™ and Kapton™. A different polymer can be used for each impervious polymer layer or the same polymer can be used for both layers.

An alternative construction of the acoustic window includes a layer of elastomer having opposed surfaces and only a layer of an impervious polymer arranged on an upper surface of the elastomer layer which is designed to be exposed to the ambient atmosphere, i.e., come into contact with the patient. In this case, the elastomer layer is attached directly to the upper surface of the transducer array, possibly by heat, pressure and optionally primers and/or adhesives. Also, the polymer layer would preferably be sealed with an impervious seal to the housing of the probe into which the transducer assembly is installed in order to prevent ingress of solvent into the probe.

Another alternative construction of the acoustic window is to provide only a polymer the layer between the elastomer layer and the transducer array in which case the upper surface of the elastomer layer is exposed. In yet another alternative construction, the acoustic window consists of only the elastomer layer, without any covering polymer layers.

An ultrasound probe in accordance with the invention includes a housing defining a cavity extending inward from an opening in a peripheral surface, a transducer array as described above arranged in the cavity of the housing to produce ultrasound beams and an acoustic window attached directly to the transducer array such that the transducer array and the acoustic window form an integral unit. The housing may be in any form of a medical imaging device including in the form of a housing of a transesophageal echocardiographic ultrasound probe or transnasal probe, or a nose for a transthoracic, intracavity or intraoperative probe. The combination of the housing and the cushion of the integral window further serve to protect the array from impact (biting by the patient in particular).

A flexible seal is interposed between the acoustic window and the housing to seal the cavity and prevent the entry of fluids into the cavity. The flexibility of the seal also enables it to absorb impacts into the acoustic window without breaking.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1 is a cross-sectional view of a portion of a first embodiment of a generic ultrasound probe in accordance with the invention taken through the transducer assembly.

FIG. 2 is an enlarged view of the section designated A in FIG. 1.

FIG. 3 is a cross-sectional view of a portion of a second embodiment of a generic ultrasound probe in accordance with the invention taken through the transducer assembly.

FIG. 4 is a cross-sectional view of a portion of a third embodiment of a generic ultrasound probe in accordance with the invention taken through the transducer assembly.

FIG. 5 is a cross-sectional view of a portion of a fourth embodiment of a generic ultrasound probe in accordance with the invention taken through the transducer assembly.

FIG. 6 is a cross-sectional view of a portion of a fifth embodiment of a generic ultrasound probe in accordance with the invention taken through the transducer assembly.

FIG. 7 is a cross-sectional view of a portion of a sixth embodiment of a generic ultrasound probe in accordance with the invention taken through the transducer assembly.

FIG. 8 is a cross-sectional view of a portion of a prior art ultrasound probe taken through the transducer assembly.

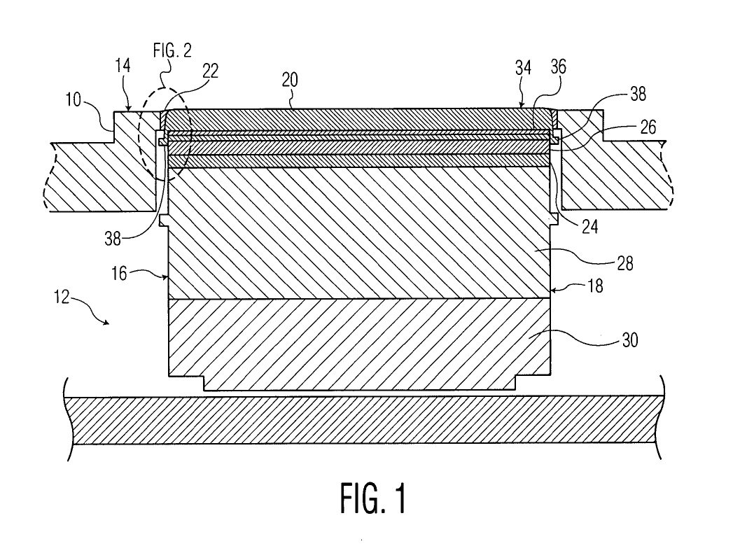

### DETAILED DESCRIPTION OF THE INVENTION

Referring to the accompanying drawings wherein like reference numerals refer to the same or similar elements, an ultrasound probe in accordance with the invention includes a housing 10 defining a cavity 12 extending inward from an opening in a peripheral surface 14 and a transducer assembly 16 arranged in the cavity 12. Housing 10 can be shaped in the form of any type of TEE, transthoracic, intracavity or transnasal probe. Housing 10 and the transducer assembly 16 in accordance with the invention can also be used in any imaging device in the medical field.

Transducer assembly 16 includes a transducer array 18 and an acoustic window 20 attached or bonded directly to the transducer array 18. The direct attachment or bonding of the acoustic window 20 to the transducer array 18 may be accomplished through the use of an adhesive or other suitable means known in the art. By attaching the acoustic window 20 directly to the transducer array 18, a transducer assembly with an integral acoustic window is formed and an acoustic window separate from the transducer array is not required, as in the prior art. Accordingly, it becomes possible to construct an ultrasound probe by providing a housing with an opening leading into a cavity in the housing, placing the entire transducer assembly 16 into the cavity and then sealing the transducer assembly 16 to the housing. The acoustic window is thus fixed to the transducer assembly and not to the housing.

By bonding the acoustic window 20 directly or intimately to the transducer array 18, the presence of a layer of lubricant between the transducer array and the acoustic window is avoided (see lubricant layer 118 in the prior art construction shown in FIG. 8). The absence of an interposition between the transducer array 18 and the acoustic window 20 improves acoustic performance. It also eliminates significant operational problems that may arise with the prior art ultrasound probe as a result of leakage of the lubricant caused by deficient construction and maintenance of the lubricant layer.

Another advantage is that the shape of the housing 10 of the probe and specifically the shape of a tip of the housing 10 in which the transducer assembly 16 is situated can be better designed for tip contact and patient intubation, without the size and space constraints necessitated for example by the presence of a rotating gear as in the prior art (see FIG. 8). Moreover, prior art acoustic windows fixed to the housing require mounting accommodations on the housing around the transducer array, e.g., a surrounding ledge to which the acoustic window is fixed. By attaching the acoustic window to the transducer array as in the invention, the space required for the mounting accommodations can be reduced.

To prevent fluids which come into contact with the acoustic window 20 from entering into the interior of the housing 10 during use of the ultrasound probe, a seal 22 is arranged between the housing 10 and the acoustic window 20. The seal 22 is made of a flexible material and should be sufficiently impermeable to fluids and materials impervious to fluids such as those used during an ultrasound examination are known to those skilled in the art.

The flexible seal 22 may be matched with the durometer of the acoustic window 20 and will thus conform to the shape of the acoustic window 20 if the acoustic window 20 is impacted. By contrast, the interface or seal between acoustic windows and housings of ultrasound probes in the prior art is stiff so that the seal will often break when the window is impacted resulting in a potential electrical safety risk.

Transducer array 18 includes a layer of piezoelectric material 24, an acoustic matching section 26 adjacent the active surface of the piezoelectric layer 24 and a backing layer 28 on the opposite side of the piezoelectric layer 24 from the acoustic matching section 26. A heatsink 30 may be arranged under or within the backing layer 28. The acoustic matching section 26 may contain one or more acoustic matching layers. The acoustic window 20 is attached to the upper surface of the acoustic matching section 26.

The transducer array 18 is preferably constructed as a matrix array for which it is not necessary to provide lateral focus through the use of a lens or structure to cause rotation thereof in order to obtain multi-planar and volumetric, three-dimensional views of the body parts being imaged. Thus, the transducer array 18 is fixed and non-rotatable relative to the housing 10 of the ultrasound probe. However, the same effect of rotation of the array element in the prior art (see FIG. 8 and the discussion above) is obtained electronically via control of the transducer elements of the transducer array 18.

To this end, transducer elements in the piezoelectric layer 24 are independently addressable and are connected to an integrated circuit which is connected in turn to a circuit board. The transducer elements may be segmented into (or designated as) transmit sub-arrays and receive sub-arrays. Each transmit sub-array may be connected to a respective intra-group transmit pre-processor which is connected to a respective transmit beamformer channel. Each receive sub-array may be connected to a respective intra-group receive pre-processor which is connected to a respective receive beamformer channel. Control of the sub-arrays is obtained by a control processor in a manner known in the art, for example, as disclosed in U.S. Pat. No. 6,572,547, the entire contents of which are incorporated by reference herein.

A matrix array will therefore be considered as a type of transducer array which is capable of generating multi-planar images on an object by appropriate electronic control of transducer elements of the transducer array.

Alternatively, a conventional two-dimensional array in which the transducer elements themselves are curved to provide the focus could be used in combination with the acoustic window 20. Furthermore, in another alternative use, the acoustic window 20 could be bonded on top of a conventional lens material to form a window or standoff.

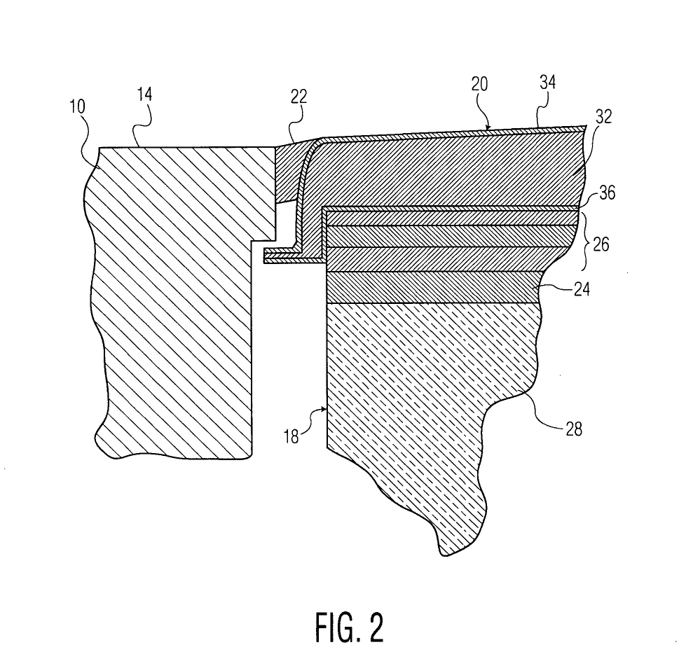

Referring now to FIG. 2, in accordance with the invention, the acoustic window 20 includes an elastomer layer 32 sandwiched between upper and lower polymer layers 34, 36, respectively, with the upper polymer layer 34 defining an outer, exposed surface of the transducer assembly 16. The lower polymer layer 36 is attached directly to the upper surface of the acoustic matching section 26 of the transducer array 18. As noted above, the absence of a gap between the transducer array 18 and the acoustic window 20, specifically between the lower polymer layer 36 and the acoustic matching section 26, avoids the need to provide an oil/lubrication layer as in the prior art embodiment shown in FIG. 8.

The elastomer layer 32 is sandwiched between the two polymer layers 34,36, e.g., thin films of polymer, and the polymer layers 34, 36 and the elastomer layer 32 are bonded to one another. The polymer layers 34,36 will therefore not separate from the elastomer layer 32 during mechanical stress or environmental cycling. The elastomer layer may be made of PEBAX™, SBS (styrene-butadiene-styrene) or SEBS (styrene-ethylene-butylene-styrene) or other suitable materials known to those in the art.

Bonding of the polymer layers 34,36 to the elastomer layer 32 can be accomplished in several ways. For example, the elastomer layer 32 can be primed and/or heated, such that it acts as an adhesive to cause the polymer layers 34,36 to be bonded thereto. In addition to or instead of priming the elastomer layer 32, it is possible to provide adhesive between each polymer layer 34,36 and the elastomer layer 32.

Bonding of materials directly to an elastomer is often problematic. Thus, in the invention, by providing the lower polymer layer 36, the transducer array 18 can be bonded more easily to the lower polymer layer 36 than to the elastomer layer 32. On the other hand, providing the upper polymer layer 34 protects the elastomer layer 32 from scratches and other types of mechanical damage and also creates a barrier that eliminates chemical susceptibility.

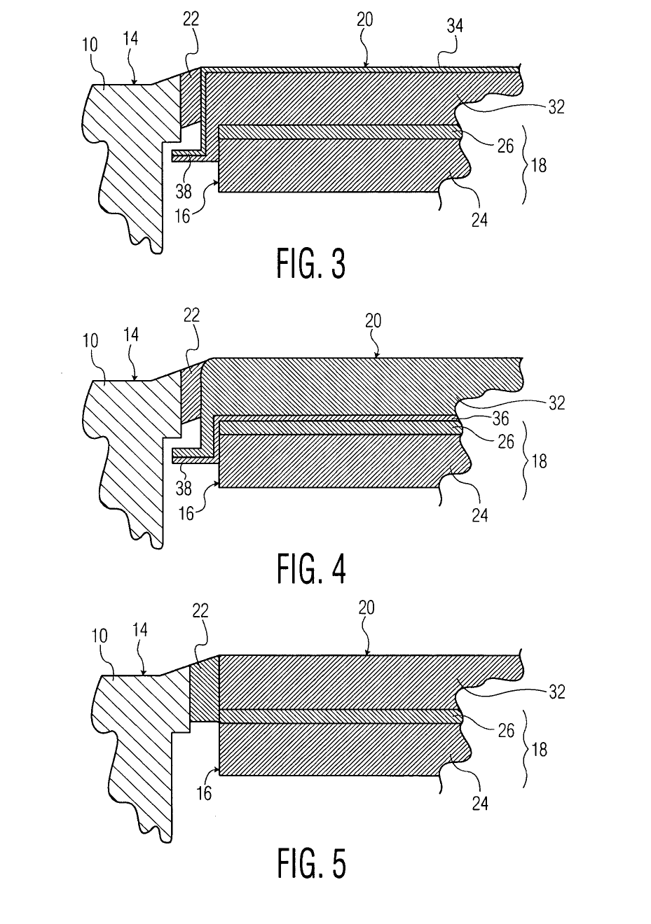

Although the embodiment of the acoustic window 20 shown in FIG. 2 includes both an upper polymer layer 34 and a lower polymer layer 36, it is possible to construct an acoustic window without the lower polymer layer 36. In this embodiment, shown in FIG. 3, the elastomer layer 32 is bonded directly to or formed directly on the upper surface of the transducer array 18, i.e., the upper surface of the acoustic matching section 26 of the transducer array 18.

The elastomer is a moldable material and therefore lends itself nicely to the production of acoustic windows having various shapes and sizes. Elastomers such as PEBAX™ can also be blended with polyethylene or other materials to tailor its properties. It is available in a range of durometers, several of which are appropriate for use in an acoustic window.

The polymer layers 34, 36 may each be made from any type of impervious polymer which preferably has a negligible acoustic impact including, but not limited to, polyethylene, Mylar™ and Kapton™. A different polymer can be used for each polymer layer 34, 36 if desired or the same polymer can be used for both polymer layers 34,36.

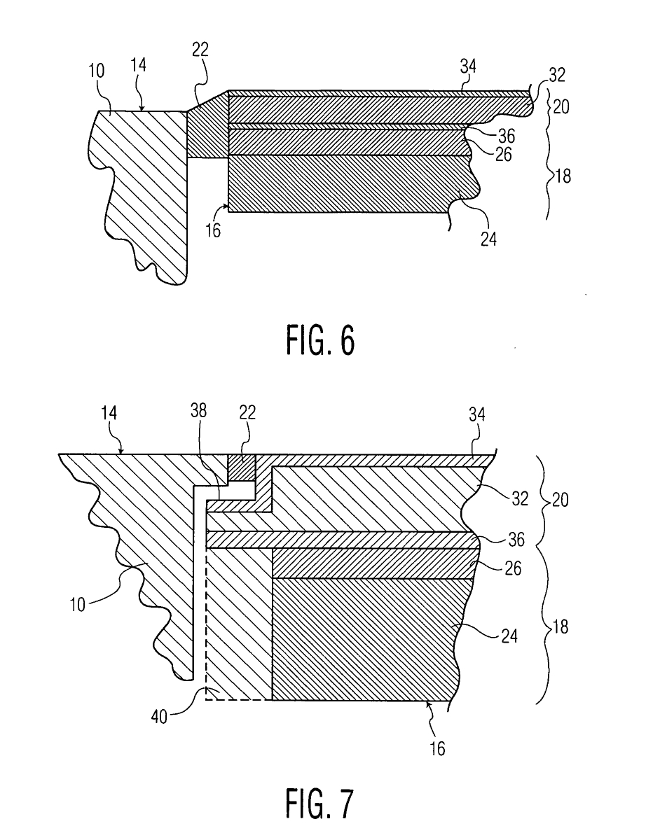

The acoustic window 20 may have a larger cross-sectional area than the acoustic matching section 26 so that a portion of the acoustic window 20 is situated alongside the acoustic matching 26. The lower polymer layer 36 is therefore bonded to the lateral edge of the acoustic matching section 26 (see FIG. 2) or may have a cross-sectional area which is substantially the same as the acoustic matching section 26 (see FIGS. 5 and 6). The acoustic window 20 has an extended section 38 alongside an upper portion of the transducer array 18 which serves to create a convoluted path (in combination with the peripheral surface of the cavity of the housing 10 into which the transducer assembly 16 is placed) to prevent fluids from entering into interior of the housing 10. This improves the electrical safety of the probe. Further, the presence of the upper polymer layer 34 provides an easy bonding of the acoustic window 20 to the seal 22.

By covering at least the portion of the elastomer layer 32 which would otherwise be exposed to the surrounding environment with the upper polymer layer 34, the outer, exposed surface of the acoustic window 20 is defined by the upper polymer layer 34 and therefore disinfectants such as isopropyl alcohol come into contact with the upper polymer layer 34 and do not come into contact with the elastomer layer 32. This avoids the problems which arise when the elastomer layer 32 comes into contact with such disinfectants.

The acoustic window 20 described above can be used in various types of ultrasound probes having transducer arrays which do not require focussing. For example, the acoustic window can be formed in a transesophageal echocardiographic (TEE) ultrasound probe, transnasal ultrasound probe, transnasal echocardiograph ultrasound probe, an intraoperative ultrasound probe or an intracavity ultrasound probe.

It is also envisioned that the acoustic window 20 is formed without the upper polymer layer 34, i.e., with only a single lower polymer layer 36 between the transducer array 18 and the acoustic window 20 (as shown in FIG. 4), or without both the upper and lower polymer layers 34,36 (as shown in FIG. 5 with the elastomer layer 32 being formed directly on the transducer array 18 as discussed above). In this case, since the elastomer layer 32 is exposed to the surrounding environment, the use of disinfectants which are not compatible with the elastomer would be prohibited. These designs could also be used for single-use devices.

FIG. 6 shows an embodiment wherein the acoustic window 20 includes the elastomer layer 32 and the upper and lower polymer layers 34,36. In this embodiment, as well as in the embodiment shown in FIG. 5, the acoustic window 20 does not extend beyond the lateral edges of the transducer array 18. Thus, the length and width of the transducer array 18 and the acoustic window 20 are substantially the same, i.e., they have the same cross-sectional area. The seal 22 thus engages the acoustic window 20 and a part of the transducer array 18.

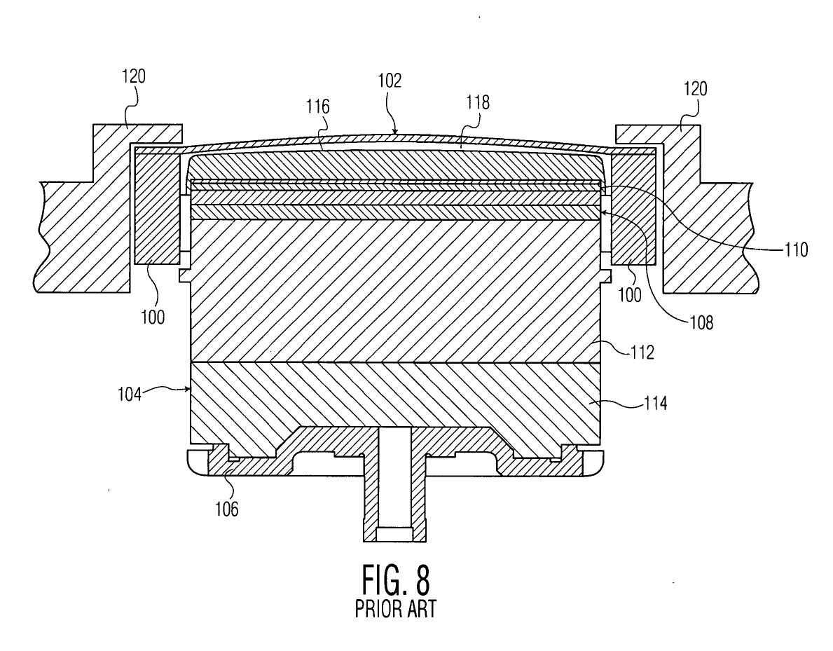

FIG. 7 shows another embodiment wherein the acoustic window 20 includes the elastomer layer 32 and the upper and lower polymer layers 34,36. In this embodiment, the acoustic window 20 does not have an extended portion 38 alongside the transducer array 18 and thus is entirely above the transducer array 18. That is, as shown in FIGS. 2-4, a portion of the acoustic window 20 is alongside the acoustic matching section 26 of the transducer array 18. By appropriate construction of the housing 10, it is possible to create a convoluted path between the housing 10 and the acoustic window 20 to prevent fluids from entering into interior of the housing. To support the portion of the acoustic window 20 extending laterally beyond the transducer array 18, an optional support 40 (shown in dotted lines) may be provided.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. A transducer assembly for an ultrasound probe, comprising:
a transducer array comprising a plurality of transducer elements; and
an acoustic window attached directly to said transducer array such that said transducer array and said acoustic window form an integral unit.

2. The transducer assembly of claim 1, wherein said transducer array comprises a layer of piezoelectric material having an active surface and defining said transducer elements, an acoustic matching section having a lower surface adjacent the active surface of said piezoelectric material and an opposed upper surface and a backing layer arranged on an opposite side of said piezoelectric material from said acoustic matching section.

3. The transducer assembly of claim 2, wherein said acoustic window is attached to said upper surface of said acoustic matching section.

4. The transducer assembly of claim 1, wherein said acoustic window comprises an elastomer layer having first and second opposed surfaces, a first layer of an impervious polymer arranged on said first surface of said elastomer layer and a second layer of an impervious polymer arranged on said second surface of said elastomer layer such that said elastomer layer is sandwiched between said first and second polymer layers.

5. The transducer assembly of claim 4, wherein a lower surface of said first polymer layer is attached to said transducer array and an upper surface of said second polymer layer defines an exposed surface of the transducer assembly.

6. The transducer assembly of claim 4, wherein said elastomer layer includes an elastomer selected from a group consisting of PEBAX™, SBS and SEBS.

7. The transducer assembly of claim 4, wherein said first and second polymer layers are thin polymer films having negligible acoustic impact.

8. The transducer assembly of claim 1, wherein said acoustic window comprises a layer of elastomer having a lower surface attached directly to said transducer array and an upper surface, and a layer of an impervious polymer arranged on said upper surface of said elastomer layer, said upper surface of said polymer layer defining an exposed surface of the transducer assembly.

9. The transducer assembly of claim 1, wherein said acoustic window comprises a layer of elastomer having an exposed upper surface defining an exposed surface of said transducer assembly and a lower surface, and a layer of an impervious polymer arranged on said lower surface of said elastomer layer and attached to said transducer array such that said polymer layer is interposed between said transducer array and said elastomer layer.

10. The transducer assembly of claim 1, wherein said acoustic window comprises a layer of elastomer having a lower surface attached directly to said transducer array and an exposed upper surface defining an exposed surface of said transducer.

11. The transducer assembly of claim 1, wherein said transducer elements are independently-addressable or curved.

12. An ultrasound probe, comprising:
a housing defining a cavity extending inward from an opening in a peripheral surface;
a transducer array arranged in said cavity of said housing to produce ultrasound beams, said transducer array comprising a plurality of transducer elements which generate ultrasound beams in various planes and volumes; and
an acoustic window attached directly to said transducer array such that said transducer array and said acoustic window form an integral unit.

13. The ultrasound probe of claim 12, wherein said housing is in the form of a housing of a transesophageal echocardiographic probe, a housing of a transnasal probe, a nose of a transthoracic probe, a nose of an intracavity probe or a nose of an intraoperative probe.

14. The ultrasound probe of claim 12, further comprising a flexible seal interposed between said acoustic window and said housing to seal said cavity.

15. The ultrasound probe of claim 12, wherein said transducer array comprises a layer of piezoelectric material having an active surface and defining said transducer elements, an acoustic matching section having a lower surface adjacent the active surface of said piezoelectric material and an opposed upper surface and a backing layer arranged on an opposite side of said piezoelectric material from said acoustic matching section, said acoustic window being attached to said upper surface of said acoustic matching section.

16. The ultrasound probe of claim 12, wherein said acoustic window comprises an elastomer layer having first and second opposed surfaces, a first layer of an impervious polymer arranged on said first surface of said elastomer layer and a second layer of an impervious polymer arranged on said second surface of said elastomer layer such that said elastomer layer is sandwiched between said first and second polymer layers.

17. The ultrasound probe of claim 16, wherein a lower surface of said first polymer layer is attached to said transducer array and an upper surface of said second polymer layer defines an exposed surface of the transducer assembly.

18. The ultrasound probe of claim 16, wherein said elastomer layer includes an elastomer selected from a group consisting of PEBAX™, SBS and SEBS.

19. The ultrasound probe of claim 12, wherein said acoustic window comprises a layer of elastomer having a lower surface attached directly to said transducer array and an exposed upper surface, and a layer of an impervious polymer arranged on said upper surface of said elastomer layer.

20. The ultrasound probe of claim 12, wherein said acoustic window comprises a layer of elastomer having an exposed upper surface and a lower surface, and a layer of an impervious polymer arranged on said lower surface of said elastomer layer and attached to said transducer array such that said polymer layer is interposed between said transducer array and said elastomer layer.

21. The ultrasound probe of claim 12, wherein said acoustic window comprises a layer of elastomer having a lower surface attached directly to said transducer array and an exposed upper surface.

22. The ultrasound probe of claim 12, wherein said transducer elements are independently-addressable or curved.
